(19) United States
(12) Patent Application Publication
(10) Pub. No.: US 2003/0125548 A1
(43) Pub. Date: Jul. 3, 2003

(54) Molecules derived from mechanism based drug design

(76) Inventors: Dorit Arad, Dallas, TX (US); Arthur P Bollon, Dallas, TX (US); David c Young, Flower Mound, TX (US); Bradley W Poland, Groton, CT (US); Andrew S Peek, Williamsburg, IA (US)

Correspondence Address: Eugenia S Hansen, Sidley Austin Brown & Wood, Suite 3400, 717 North Harwood, Dallas, TX, 75201, US

(21) Appl. No.: 10/149,087

(22) PCT Filed: Dec. 22, 2000
(86) PCT No.: PCT/US00/34964

(51) Int. Cl. G06G 7/48; G06G 7/58; C07D 279/12; C07D 241/04; C07D 265/30

(52) U.S. Cl. 544/59; 703/12; 544/176; 544/386

(57) ABSTRACT

A method for identifying molecules and designing novel pharmaceuticals is disclosed. The disclosed method of novel pharmaceutical design identifies a novel chemical group that triggers a mechanism of action for an identified reaction, thereby avoiding the various structural complexities associated with the application of the structure-based drug design. Additionally, examples of potential therapeutics identified by the mechanism based drug design method are provided.

### TECHNICAL FIELD OF THE INVENTION

The field of the invention generally relates to a mechanism-based drug design system and methodology for identifying small molecules, in particular those that can be developed into pharmaceutical compounds.

### BACKGROUND OF THE INVENTION

The search for pharmaceutical agents to combat diseases is an ongoing process. Several approaches to identify pharmaceutical agents are currently used: (i) random drug screening, (ii) rational drug screening, and (iii) structure based drug design. Each method has its own set of advantages and disadvantages depending on the amount of information and resources available.

Random drug screening is the process by which a large number of molecules are tested in an assay to determine activity Although very little information about the molecular target or its agonist is required to successfully use this method, the investment of time and resources is high because of the extensive number of compounds needed to be synthesized and assayed for activity. As a consequence, random drug screening is practiced almost exclusively by large commercial entities, although implementation of robotics for high throughput screening and synthesis of chemical libraries by combinatorial methods have made this a less expensive process (see Gallop, et al., “Applications of combinatorial technologies to drug discovery, background and peptide combinatorial libraries,” J Med Chem 37: 1233-50 (1994)). However, the tradeoff for easier screening and greater availability of compounds is the identification of more lead compounds, which must undergo further experimentation to determine clinical viability, thereby increasing the expense of the entire process. Although software is being developed to analyze drug leads for pharmacokinetic profiles, there exists a need for drug discovery methods that begin with a smaller number of drug leads.

To effectively use the rational drug screening method, a ligand providing desired effects must be known and an effective assay developed. In this method, molecules with similar structural or chemical properties as the target ligand are synthesized and screened in an activity assay. The advantage of this method over the random drug screening method is that potentially fewer compounds must be screened. The disadvantage, however, is that more information must be known about the ligand, and preferably its target site, before a lead compound can be identified.

A method requiring even more a priori knowledge before it can be used effectively is structure-based drug design (SBDD). The premise of this method is that potent pharmaceuticals possess significant structural and chemical compatibility to their target site. Thus, an understanding of the principles of molecular recognition in the target protein-ligand complexes is important for employing SBDD. To use SBDD, the target must be identified and its three-dimensional structure solved. (Hans-Joachim Bohm and Gerhard Klebe, “What can we learn from molecular recognition in protein-ligand complexes for the design of new drugs?” Agnew Chem nt EdEngl 35: 2588-2614 (1996)). The application of SBDD has already assisted in providing several successfl pharmaceutical products. For example, the enzyme inhibitor dorzolamide, which has been approved by the FDA for treating glaucoma, was the first approved pharmaceutical where the methods of structure-based drug design significantly contributed to its discovery.

Computers and software play pivotal roles in SBDD because they generate graphic representations of three-dimensional structures of proteins and protein-ligand complexes to identify essential interactions. In addition, software that searches for additional binding sites not used by previously known ligands can also be used. For example, possible ligand binding sites might be positions where hydrogen bonds can be formed with an enzyme(s), or hydrophobic pockets in the enzyme structure. Moreover, a number of computational tools have been described to select putative ligands and to predict their interactions with the protein. In general, these tools for ligand design can be divided into the categories that follow:

1) analysis of the protein structure,

2) ligand docking and three-dimensional database searching,

3) de novo ligand design,

4) assessment of the ligand binding affinity, and

5) analysis of the ligands electronic and conformational properties of the ligand.

Several of these categories for designing ligands using a computer are described in the Agrafiotis patents (U.S. Pat. No. 5,684,711 and 5,901,069). In Agrafiotis, the computer generates chemicals with defined physical, chemical and/or bioactive properties. For example, the computer employs a process wherein each iteration of the process includes: (1) the generation of a diverse chemical library, (2) analysis of such compounds in the chemical library having desired properties, (3) the implementation of structure-property data to select compounds to be synthesized, and (4) the generation of new synthesis instructions to control the synthesis of the diverse chemical library for the next iteration.

More specifically, each cycle of the process described in the Agrafiotis patents begins with a robotically synthesized diverse chemical library comprising numerous chemical compounds, which are then analyzed to obtain structure-activity/structure-property data. The data is stored in a database that contains the structure-activities and structure-properties of previously synthesized compounds. The computer evaluates the structure-activity and structure-property data of chemical compounds obtained from previous iterations and constructs structure-activity/structure-property models that substantially conform to the observed data. The computer, using a reagent database, can then generate compounds to be tested and evaluated for having the desired pharmaceutical properties.

Additional patents involving the use of computer simulation for a rational drug design methodology are the Balaji patents (U.S. Pat. Nos. 5,579,250 and 5,612,895). The Balaji patents describe a method of rational drug design capable of identifying bioactive peptidomimetics that can be effectively used as drugs. The method includes: (1) the use of a computer to simulate the most probable conformation of a given polypeptide, (2) the simulation and design of the most probable conformation of a chemically modified analog of a peptide, (3) the evaluation of the activity of the synthesized chemically modified analog, and (4) the potential design of a suitable peptidomimetic based on the confirmation of the synthesized chemically modified analog.

To predict the most probable tertiary structure of the peptide, the Balaji patents introduce the “ab initio” method, which is used to simulate a tertiary structure of a protein in the absence of physical or chemical data. The results of the “ab initio” calculations indicate the most probable conformation of such a peptide. Additionally, the Balaji patents introduce an analytical tool referred to as the “Balaji Plot,” which is automatically generated when performing the “ab initio” method. The “Balaji Plot” can assist in determining the sequences of flexible and rigid areas in peptides or peptidomimetics, and provides insight as to how rigid, constrained or flexible peptide analogs should be modeled by computer programs.

Although SBDD has emerged as a useful methodology to identify potential molecular reactions between a target site and a proposed pharmaceutical, the use of SBDD has been limited by the large amount of a priori knowledge needed to effectively use it. The novel design methodology described herein focuses on the mechanism of action of proteins (eg. enzymes) to determine the “trigger” mechanism that drives a particular reaction. Pharmaceuticals can be designed from identified trigger mechanisms. By designing a compound around the trigger mechanism of action rather than molecular interactions at a defined loci, potent, low molecular weight target-specific compounds can be rapidly produced.

Examples of such compounds are also disclosed.

### BRIEF DESCRIPTION OF THE DRAWINGS

FIG. 1 depicts a free energy diagram illustrating the action of a substrate—enzyme complex (-), an inhibitor—enzyme complex ( - - - - ), and an enhancer—enzyme complex ( - - - - - ).

FIG. 2 depicts a flow chart for determining the trigger mechanisms for enzymes as they relate to a particular substrate.

### SUMMARY OF THE INVENTION

One aspect of the invention includes a molecule comprising a minimal quantity of atoms to trigger a desired reaction wherein the molecule is designed by the steps of: (a) determining the minimal quantity of atoms to trigger a desired reaction to occur, the minimal quantity of atoms collectively comprise the trigger mechanism for the desired reaction; and (b) positioning the minimal quantity of atoms on a ligand wherein the molecule can trigger the desired reaction independently from the naturally occuring desired reaction.

In yet another aspect of the invention includes a molecule comprising a minimal number of atoms for inhibiting a mechanism of action of a given reaction wherein the molecule is designed by the steps of: (a) determining the trigger mechanism of the given reaction; and (b) positioning the minimal number of atoms on a ligand wherein the ligand comprises an atom that functions as a poor leaving group when the molecule interacts with a reactant of the given reaction, thereby causing the molecule to inhibit the mechanism of action of the given reaction. Examples of such molecules are as follows:

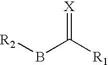

wherein B can be any basic group,

R₁ and R₂ can be any functional group; and

x can be any atom or group that is more electronegative than sp² carbon;

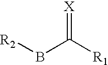

wherein B can be any basic group;

R₁ and R₂ can be any functional group; and

x can be any atom or group that is more electronegative than sp² carbon;

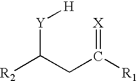

wherein x can be any atom or group that is more electronegative than sp² carbon; R₁ and R₂ are any functional group; and y is any proton donating group; and

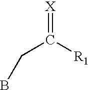

wherein B can be any basic group; R₁ and R₂ can be any functional group; and x can be any atom or group that is more electronegative than sp² carbon.

In yet another aspect of the invention a system for developing a small molecule is provided wherein the system comprises: (a) a method to compare the stabilization energies of a given reaction between a catalytic residue of an enzyme to residues of a substrate to determine the trigger mechanism for the given reaction; (b) a minimal amount of atoms comprising the trigger mechanism of the given reaction; and (c) a ligand wherein said minimal amount of atoms are positioned on said ligand to form said molecule. Examples of such molecules include the minimum quantity of atoms to either inhibit or enhance the given reaction.

In yet another aspect of the invention a method for determining the trigger mechanism of a given reaction is provided wherein the method uses a matrix involving the steps of: (a) performing ab initio calculations on a class of enzymes to compare a reactive residue associated with a catalytic site common to the class of enzymes to a reactive residue with one or more substrates associated with the class of enzymes; (b) using the information generated from step (a) and performing further ab initio calculations on a subclass of enzymes by comparing further reactive residues associated with the catalytic site common to the subclass of enzymes with one or more substates associated with the subclass of enzymes; (c) using the information generated from step (b) and performing further ab initio calculations on a single enzyme from the subclass of enzymes by comparing all reaction residues associated with the catalytic site of the enzyme with one or more substrates associated with the enzyme; and (d) using the information generated from step (c) to determine the trigger mechanism for the enzyme.

In yet another aspect of the invention a method for determining a trigger mechanism for a given reaction is provided comprising the steps of: (a) performing ab initio calculations to determine the stability of each potential interaction between each active site residue of an enzyme and a chemical moiety of one or more substrates of said enzyme; (b) analyzing the calculated stabilization energies, wherein the most negative energies are the most reactive species and the most positive energies are the least reactive species; and (c) combining the chemical moieties that were calculated to be the most reactive, or the least reactive, with each corresponding catalytic residue on a compound scaffold.

In yet another aspect of the invention a method for deteriming the trigger mechanism of a given reaction is provided comprising the steps of: (a) aligning a protein sequence of an enzyme with other proteins and determining by homology the general class of the enzyme; (b) performing biochemical assays to determine the reaction performed by the enzyme; (c) performing site-directed mutagenesis of conserved amino acids and determining if the mutated residues act in the reaction mechanism; (d) determining the mechanism of catalytic action for the enzyme; and (e) performing ab initio calculations to determine the stability of each potential interaction between each active site residue of the enzyme and a chemical moiety of one or more substrates of said enzyme.

In yet another apsect of the invention a method for designing a small molecule that interacts with the active site of a certain enzyme is provided comprising the steps of: (a) aligning the protein sequence of the enzyme with other proteins and determining by homology the general class of enzyme; (b) performing biochemical assays to determine the reaction performed by the enzyme; (c) performing site-directed mutagenesis of conserved amino acids and determining if the mutated residues act in the reaction mechanism; (d) determining the mechanism of catalytic action for the enzyme; (e) performing ab initio calculations to determine the stability of each potential interaction between each active site residue of the enzyme and a chemical moiety of one or more substrates of said enzyme; (f) analyzing the calculated stabilization energies, wherein the most negative energies are the most reactive species and the most positive energies are the least reactive species; and (g) combining the chemical moieties that were calculated to be the most reactive, or the least reactive, with each corresponding catalytic residue on a compound scaffold. Examples of such molecules contain a minimum quanty of atoms that can inhibit or enhance a given reaction.

In yet another aspect of the invention a molecule comprising a minimal quantity of atoms to inhibit the activation of telomerase is provided wherein said molecule comprises a poor leaving group

In yet another aspect of the invention a molecule comprising a minimal quantity of atoms to inhibit the activation of mycothiol s-conjugate amidase is provided wherein said molecule comprises a poor leaving group.

### DETAILED DESCRIPTION OF THE INVENTION

The mechanism-based drug design technology (Quantum Core Technology™) of the present invention is a system and method for obtaining novel molecules that can function as pharmaceuticals or undergo modification to act as pharmaceuticals. The molecules obtained by this technology are small and specific to the target of interest, making them more likely to possess low toxicity. Because the majority of the designed compounds are based on the trigger mechanism (as discussed below) of a specific enzyme, the enzyme is not likely to evolve resistance to the molecules. The disclosed invention can also be used to design enzyme—inhibiting as well as enzyme—enhancing compounds.

The method and system of the invention takes into account the four steps of enzymatic catalysis. First, in the inactive state of the enzyme, the spatial conformation of the catalytic residues do not allow interaction with another. Next, the substrate interacts with the enzyme to form an enzyme substrate complex (E−S complex). The E−S complex consists of numerous noncovalent interactions between the active site residues and the substrate. As the number of such nonconvalent interactions increases, the greater the E−S binding energy becomes. Third, the formation of the E−S complex activates the catalytic machinery of the enzyme by inducing a change in the spatial conformation of the catalytic residues. This change allows the catalytic residues to react with the substrate and catalyze the chemical reactions, a process referred to herein as “triggering.” For proteins, the binding of a specific molecule to a protein triggers its transformation from an inactive state to an active state in which it can interact specifically with other proteins, DNA or RNA. Finally, after the substrate is converted to product and released, the enzyme returns to the inactive confirmational state, without having been consumed or altered in the reaction.

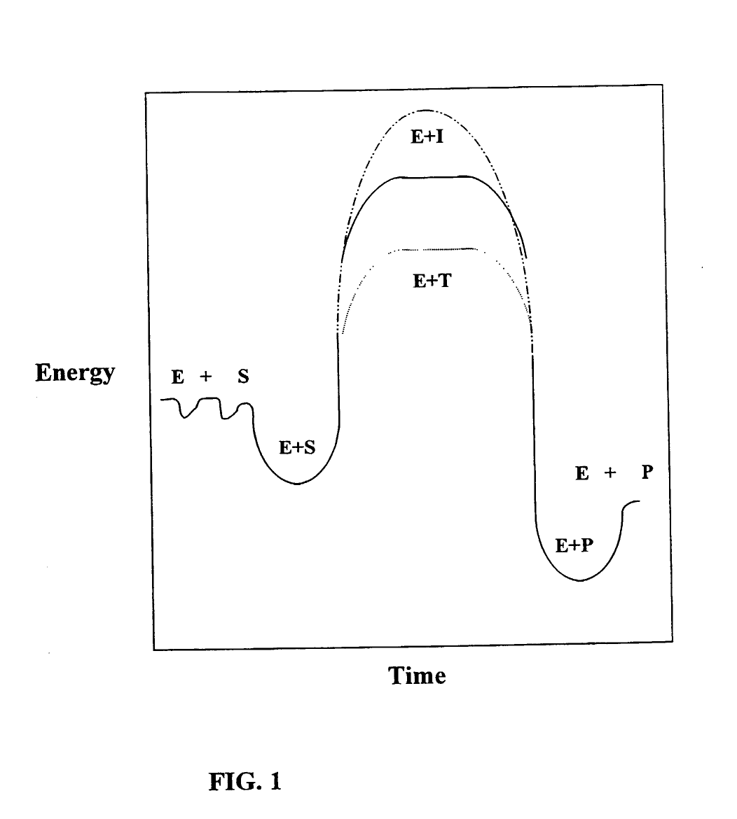

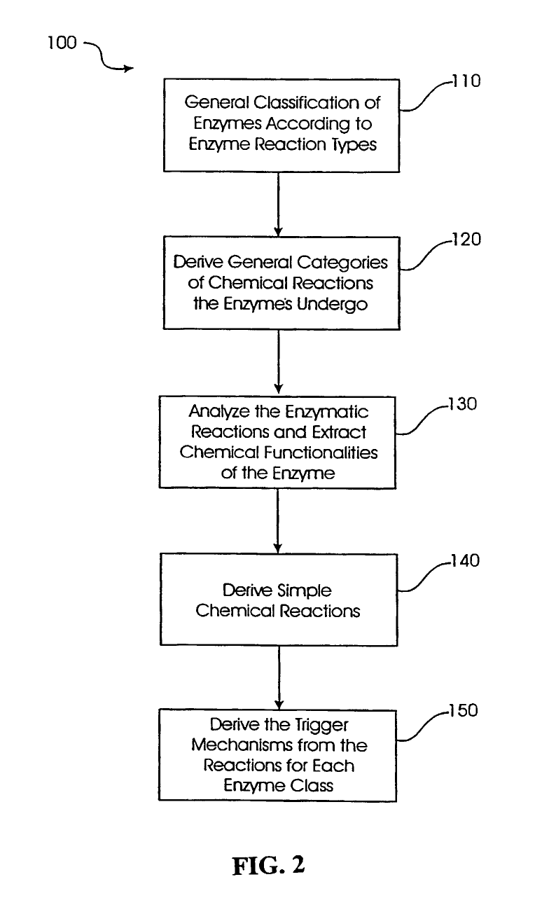

To further illustrate the invention described herein, FIGS. 1-2 are provided to assist in teaching the invention. Each figure is, however, merely for the purpose of illustrating the invention by way of example. Thus, it is to be expressly understood that FIGS. 1-2 are for illustration purposes only, and therefore not intended, in any fashion, to be a definition of the limits of the invention.

FIG. 1 is a free energy diagram that thermodynamically describes enzyme catalysis and inhibition. When an enzyme (E) is inactive, the spatial conformation of the catalytic residues do not allow interaction with one another. However, in the presence of a substrate (S), the enzyme begins to rearrange as numerous noncovalent interactions between the active site residues and the substrate form, which are indicated by the small peaks and valleys in the leftmost portion of the diagram. The binding energy between the enzyme and the substrate increases as the number of interactions increase, leading to a stabilized enzyme—substrate complex (E+S), depicted by the small valley just before the large peak in the diagram. The formation of the E+S complex allows the catalytic residues to react with the substrate and to begin the catalysis reaction by performing any of the functions that follow: spatial rearrangement, form a particular electronic state, form intramolecular hydrogen bonds, or perform an internal proton transfer. The role of the catalyst is to lower the energy activation barrier so that the reaction can proceed. After the reaction, the enzyme releases the products (P) at a lower energy state. As a hypothetical example, the E+S complex may have an energy barrier as depicted by the solid line. An enzyme inhibitor (I) increases the energy barrier needed to overcome to perform a reaction, as shown in the dashed—dotted line. A molecular trigger (T), defined herein below, decreases the energy barrier needed to overcome to perform a reaction, as shown in the dotted line.

Unlike SBDD, in which molecules are designed to bind more strongly to the enzyme or compete with the substrate for active site binding, the present invention identifies a molecule that acts to initiate the reaction of an enzyme independent from the enzyme carrying the reaction trigger. The trigger for a reaction is the internal rearrangement that must be satisfied in order for the enzyme to form the reactant complex, which will lead to a reaction along the path catalyzed by the enzyme. The direct activation of the enzyme by the molecular trigger bypasses the need for multiple noncovalent interactions that typically occur in the natural enzyme activation process. Molecular triggers will reach the reactive state more readily than the native substrate due to either the energetic or kinetic processes involved. A successful drug or compound incorporating a molecular trigger will often form this initial complex more quickly or energetically more favorably than the native substrate, but will not complete the reaction because a suitable leaving group is lacking.

The same can be applied to proteins which are not enzymes. For example, molecular triggers can be identified for reactive, non-enzymatic proteins. In this instance, a trigger can be identified that induces a conformational change leading to an allosteric effect or signal activation.

In general, the method and system of the invention can employ known information about an enzyme target. The novel system and method of the present invention is depicted at 100 in FIG. 2. FIG. 2 is a flow chart depicting the preferred steps of the methodology for determining the trigger molecules of an enzyme. These preferred steps include: (1) developing a general classification of enzymes according to their reaction types 110, (2) deriving general categories of chemical reactions that enzymes undergo 120, (3) analyzing the enzymatic reaction and extracting the role of the enzyme's chemical functionalities 130, (4) deriving simple chemical reactions 140, and (5) deriving from the reaction the trigger mechanism for each enzyme class 150. It should be noted that in most instances steps 1 and 2 can be overlooked if the enzyme being examined has previously been classified. To derive the molecular trigger, “ab initio” calculations are performed for each square of a two-dimensional matrix and the calculated stabilization energies are analyzed. An example of the two-dimensional matrix is provided at Table I.

As previously discussed, the preferred first step of the invention is to classify a target enzyme according to its reaction type, which is depicted at 110 in FIG. 2. According to the Commission on Enzymes of the International Union of Biochemistry, enzymes are currently classified into six general groups according to their performed reaction. For example, enzymes in the hydrolase class catalyze a hydrolysis reaction. The enzymes can be further classified according to their biological function and/or preferred substrates. Several databases exist that include an extensive amount of information about enzymes as they relate to their designated class. For example, see Enzymes.htm at <http://crisceb.area.na.cnr.it/angelo/petrilli/enzymesfintor.htm>or ExPASy-Enzyme.htm at http://www.expasy.ch/cgi-bin/nicezyme.pI?2.3.1.43. The enzyme reaction classifications recognized by the Commission on Enzymes of the International Union of Biochemistry are provided in Table II below.

Ascertaining the reaction classification of an enzyme can be accomplished with methods established in the art. Oftentimes, the reaction type of an enzyme is already known or can be easily identified from the published sequence of the enzyme. If this is not the case, there are methods in the art to ascertain particular aspects of an enzyme if they are not currently known. For example, the reaction an enzyme catalyzes can be determined by performing enzyme assays, which are known to those skilled in the art. (G. Sarath, R. S. de la Motte and F. W. Wagner. In Proteolytic Enzymes: A Practical Approach, ed. R. J. Beyon and J. S. Bond, IRL Press, 1990, p. 25). If the enzyme of interest has not been previously isolated and/or characterized, it can be isolated using a variety of techniques commonly practiced by biochemists. The enzyme can then be subjected to amino acid sequence analysis and the DNA sequence analyzed for the enzyme cloned with

TABLE I
Protease model nucleophiles, electrophiles and complexes

| | | 1 | 2 | 3 | 4 | 5 | 6 | 7 |
|---|---|---|---|---|---|---|---|---|
| | | 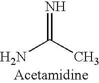 | 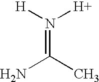 | 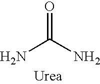 | 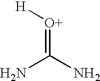 | 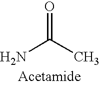 | 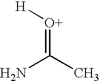 | 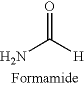 |
| 1 | OH— | 7.7 | 161.5 | 15.9 | 173.1 | 22.0 | 183.3 | 21.8 |
| 2 | H₃C—O— | −1.1 | 153.8 | 8.4 | 165.1 | 13.0 | 172.5 | 14.3 |
| 3 | 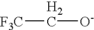 | −11.7 | 131.2 | −3.1 | 141.8 | 1.5 | 147.9 | 3.3 |
| 4 | 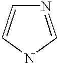 | −26.1 | 114.4 | −17.8 | 123.9 | −11.4 | 134.9 | −7.4 |
| 5 | SH— | NTC | 101.6 | NTC | 111.4 | NTC | 123.6 | NTC |
| 6 | H₃C—S— | NTC | 109.7 | −29.8 | 120.6 | NTC | 132.1 | NTC |
| 7 | 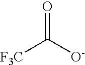 | NTC | 87.4 | NTC | 97.8 | NTC | 106.8 | NTC |
| 8 | HOH | NTC | NTC | NTC | NTC | NTC | NTC | NTC |
| 9 | H₃—OH | NTC | NTC | NTC | NTC | NTC | NTC | NTC |
| 10 | 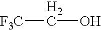 | NTC | NTC | NTC | NTC | NTC | NTC | NTC |
| 11 | 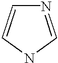 | NTC | 4.8 | NTC | 8.9 | NTC | 21.1 | NTC |
| 12 | HSH | NTC | NTC | NTC | NTC | NTC | NTC | NTC |
| 13 | H₃C—SH | NTC | NTC | NTC | NTC | NIC | −20.5 | NTC |
| 14 | 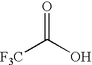 | NTC | NTC | NTC | NIC | NTC | NTC | NTC |

| | | 8 | 9 | 10 | 11 | 12 | 13 | 14 | 15 |
|---|---|---|---|---|---|---|---|---|---|
| | | 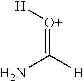 | 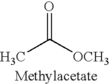 | 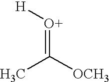 | 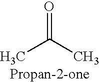 | 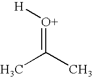 | 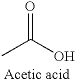 | 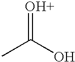 | 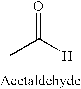 |
| 1 | OH— | 195.0 | 27.5 | 192.3 | 29.2 | 205.7 | 29.2 | 202.0 | 23.6 |
| 2 | H₃C—O— | 189.5 | 18.0 | 185.4 | 19.7 | 197.9 | 21.5 | 195.2 | 22.6 |
| 3 | 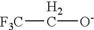 | 164.8 | 0.7 | 160.8 | 8.6 | 174.3 | 9.2 | 170.5 | 12.2 |
| 4 | 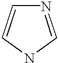 | 148.7 | −6.1 | 143.3 | −4.7 | 157.4 | −3.9 | 153.9 | −0.2 |
| 5 | SH— | 136.3 | NTC | 130.1 | NTC | 148.1 | NTC | 140.3 | NTC |
| 6 | H₃C—S— | 145.9 | NTC | 139.0 | NTC | 156.6 | NTC | 148.0 | NTC |
| 7 | 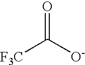 | 120.5 | NTC | 114.8 | NTC | 129.9 | NTC | 124.3 | NTC |
| 8 | HOH | NTC | NTC | NTC | NTC | NTC | NTC | NTC | NTC |
| 9 | H₃C—OH | NTC | NTC | NTC | NTC | NTC | NTC | NTC | NTC |
| 10 | 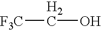 | NTC | NTC | NTC | NTC | NTC | NTC | NTC | NTC |
| 11 | 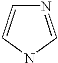 | 28.5 | NTC | 24.7 | NTC | 41.7 | NTC | 36.8 | NTC |
| 12 | HSH | NTC | NTC | NTC | NTC | NTC | NTC | NTC | NTC |
| 13 | H₃C—SH | −10.0 | NTC | −13.3 | NTC | 2.6 | NTC | −7.0 | NTC |
| 14 | 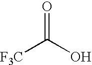 | NTC | NTC | NTC | NTC | NTC | NTC | NTC | NTC |

| | | 16 | 17 | 18 | 19 | 20 | 21 | 22 |
|---|---|---|---|---|---|---|---|---|
| | | 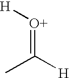 | 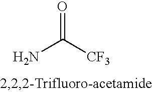 | 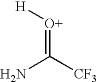 | 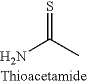 | 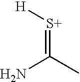 | 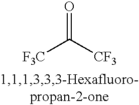 | 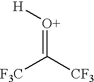 |
| 1 | OH— | 219.4 | 46.4 | 210.9 | 48.5 | 183.3 | 79.9 | 267.9 |
| 2 | H₃C—O— | 213.7 | 38.4 | 203.5 | 39.2 | 171.0 | 70.2 | 259.1 |
| 3 | 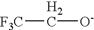 | 189.4 | 24.4 | 178.7 | 25.4 | 150.0 | 54.1 | 233.8 |
| 4 | 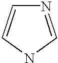 | 173.7 | 9.9 | 158.7 | 14.2 | 136.1 | 36.9 | 214.4 |
| 5 | SH— | 160.3 | −9.2 | 147.5 | −6.3 | 127.9 | 20.0 | 206.2 |
| 6 | H₃C—S— | 170.1 | −3.9 | 158.1 | 2.4 | 136.7 | 24.2 | 214.2 |
| 7 | 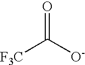 | 136.7 | NTC | 129.0 | −15.8 | 104.7 | 14.5 | 184.7 |
| 8 | HOH | NTC | NTC | NTC | NTC | NTC | NTC | 28.4 |
| 9 | H₃C—OH | 18.1 | NTC | −1.8 | NTC | NTC | NTC | 40.0 |
| 10 | 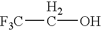 | NTC | NTC | NTC | NTC | NTC | NTC | 26.6 |
| 11 | 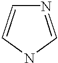 | 54.0 | NTC | 37.5 | −23.0 | 20.0 | NTC | 83.7 |
| 12 | HSH | 2.3 | NTC | NTC | NTC | NTC | NTC | 24.1 |
| 13 | H₃C—SH | 13.7 | NTC | −7.6 | NTC | NTC | NTC | 39.3 |
| 14 | 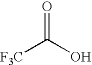 | NTC | NTC | NTC | NTC | NTC | NTC | NTC |

HF/6-31+G*
Binding in kcal/mol
NTC = no tetrahedral complex

TABLE II
Enzyme Classification by Commission on Enzymes
of the International Union of Biochemistry

| | Predicted Number of |
|---|---|
| Enzyme Classes According to Reaction-Types | Enzymes |
| 1. Oxidoreductases | 3766 |
| 2. Transferases | 4364 |
| 3. Hydrolases | 4649 |
| 4. Lysases | 1604 |
| 5. Isomerases | 576 |
| 6. Ligases | 655 |

techniques commonly known to persons practicing in the field of molecular biology (See Short Protocols in Molecular Biology, 2d. ed., John Wiley & Sons (1992), which is incorporated herein by reference. To determine the DNA sequence of the target protein, the method discussed in Wilhelm Arronage, DNA Sequencing Strategies, Automated and Advanced Approaches, ISBN 0971136832 (S. Zimmerman, ed.), which is also incorporated herein by reference, is preferred. The determined DNA and amino acid sequences of the enzyme can be compared for homology to other known sequences of classified enzymes having an identified tertiary, or preferably quaternary structure. These higher level structures of enzymes are located in the Protein Data Bank of the Brookhaven National Laboratory. If there is no available quaternary structure for the enzyme of choice, but it is highly homologous to other enzymes, the three-dimensional structure of the studied enzyme can be modeled onto the solved structure Additionally, if the enzyme is not identified in existing databases, the reaction it catalyzes can be determined by performing enzyme assays, which are known to those skilled in the art. Examples of such enzyme assays use natural or synthetic substrates for spectrophotometric or fluorimetric assays to characterize enzymes. (G. Sarath, R. S. de la Motte and F. W. Wagner. In Proteolytic Enzymes: A Practical Approach, ed. R. J. Beyon and J. S. Bopand, IRL Press, 1990, p. 25).

After an enzyme has been classified according to its reaction, the next preferred step is to derive the general category of chemical reaction the enzyme undergoes. This step occurs at 120 in FIG. 2. For example, if the enzyme is a protease, the chemical reaction would be a hydrolysis. On the other hand, if the enzyme is an amidase, the chemical reaction would be a deamination.

After categorizing the chemical reaction an enzyme undergoes, analysis to identify the mechanism of action of the enzyme is performed, which is represented as 130 in FIG. 2. The foundation for determining a reaction mechanism is based on organic chemistry and biochemistry. Furthermore, application of energetic principles (e.g., Gibb's free energy principle) and electronic properties of the various molecules involved in the enzymatic reaction can assist in determining the mechanism of action. By identifying the active site residues, one can begin to understand the enzyme's mechanism. Criteria that active site residues satisfy are listed below:

1) active site residues are present in all known examples of the particular class of enzymes;

2) Modification of the active site residues yields a debilitated enzyme;

3) Within the three dimensional structure of the enzyme, the active site residues are often closely situated; and

4) Within the three-dimensional structure of the enzyme, the active site residues are inside a groove or pocket and the orientation of the active residues allows them to interact with the substrate at a location consistent with the enzymes overall function.

Beyond identifying the active site residues, the mechanism of an enzyme can be described by a plausible chemical reaction, or series of reactions, that show how the active residues and substrate undergo a reaction consistent with the known function of the enzyme. Data that describes the kinetics of the enzyme's reaction can be analyzed to determine if it is consistent with a hypothesized mechanism. Additionally, analysis of the thermodynamics of a hypothesized reaction can indicate whether a hypothesized mechanism is valid.

“Ab initio” calculations can be used to assist in generating a model reaction. “Ab initio,” which in Latin means “first principles,” is a calculation procedure. The distribution of electrical charge, a molecular wave function, and various molecular properties can be determined using “ab initio” calculations. Examples of molecular properties include: stabilization energies, orbital energies, electronic properties, chemical properties, heats of formation, dipole moments, and intermediate stabilities. Chemical properties can also be predicted using “ab initio” calculations. Thus, “ab initio” calculations performed on a model reaction mechanism can be used to simulate the most favorable reaction mechanism of the target protein so to further define the most probable reaction (W. J. Hehre, L. Radom, P. V. R. Schleyer, and J. A. Pople. Ab Initio Molecular Orbital Theory, Wiley (1986)). The reaction having the more negative free energy value will demonstrate the more stable reaction intermediate because it requires the lowest amount of energy for the reaction to occur. In other words, the most labile reaction will possess the most negative free energy. Preferably, the “ab initio” calculations are performed with the assistance of a computer employing the Gaussian Series of Programs (University of California at San Francisco).

Once the reaction mechanism has been determined, the next preferred step is to derive the trigger molecule for an enzyme. This step is illustrated at 150 of FIG. 2. Because enzymes are classified according to their reaction type and mechanisms, the triggers for enzymes of a particular class are likely to be similar. For example, the 3C protease from the picornavirus family and human caspase are both proteases that have only cysteine and histidine as their catalytic residues and so could be triggered by similar molecules. Triggers are identified using “ab initio” calculations.

In order to analyze “ab initio” calculations to determine the molecular trigger, a two-dimensional matrix is developed to calculate the stabilization energy of the resulting products relative to the reactants. An example of such a two-dimensional matrix to determine the molecular trigger of cysteine proteases is provided in Table II. The x-axis of the matrix represents all the key chemical moieties on the catalytic residues of the enzyme in its resting state. The y-axis represents all reactive moieties of known substrates and inhibitors. The free energy for each potential interaction between two moieties is calculated using “ab initio” calculations (described above) in a reliable basis set representing consistent assumptions about the interactions. The reliable basis set is selected on the grounds that it yields energies and energy differences that are close to the experimental value.

A preferred basis set is HF/6−31+G*//HF/6−31G*, which can be obtained by using programs such as Gaussian (Guassian Corp.), Q-chem (Q-Chem), and Spartan (Wavefunction). The “ab initio” calculations can be used to determine which of the chemical functionalities are likely to be inhibitors or enhancers for a particular enzymatic reaction. For example, a strong inhibitor interacting with an enzyme will decrease the negative free energy value, thereby making the reaction thermodynamically less favorable. Unlike an inhibitor, an enhancer will bind the enzyme with a large negative free energy value. By calculating all of the possible combinations in the matrix, the combination representing the most favorable reaction for a specific reaction can be determined.

### EXAMPLE 1

### How Mechanism Based Drug Design can Identify Reactive Molecules to Modulate Serine Proteases

Proteases are part of the hydrolase class of enzymes, which are selected from the enzymatic class having the general reaction equation that follows:

ROR′+enzyme+H2O----ROH+R′OH

The protease family is selected from the enzymatic class having the general reaction equation of:

R—CONHR′+enzyme+H2O---->RCOOH+R′NH2

Generally, the protease family consist of four major classes: serine proteases, cysteine proteases, aspartic proteases, and metalloproteases, each so named for the enzyme component believed to be critical for catalysis.

Table I shows an example of a matrix constructed for determining the most probable (favorable) reaction between the reactants in the x and y axis using “ab initio” calculations. The x axis of the matrix includes all key catalytic residues of the proteases, while the y axis represents all possible substrates and inhibitors for the different types of reactions within the protease family. The resulting energies of the products are compared for inhibition and activation. Candidate inhibitors having the best stabilization energies and inhibition are selected. The matrix can be further expanded to include general acid base reactions relevant to all hydrolases. This version of the matrix includes a large variety of substrates. Examples include organic phosphates, carbohydrates, fatty acid esters, several amides, esters, epoxides, nucleic acid bases, etc. The results of “ab-initio” calculations for the different constituents of each protease provides for a large selection of potential core molecules. Again, by focusing on the stabilization energies calculated for the products of the various reactant combinations, a potential modulator can be located by selecting the products having the best stabilization energies.

For example, the triggers for serine proteases, aspartic proteases, metalloproteases, and cysteine proteases were determined using “ab initio” calculations and quantum mechanic principles as previously discussed. The triggers for the various proteases are as follows:

(1) the serine protease trigger involves the removal of a hydrogen from the serine residue, which allows the serine anion to then attack the electorphile as illustrated below:

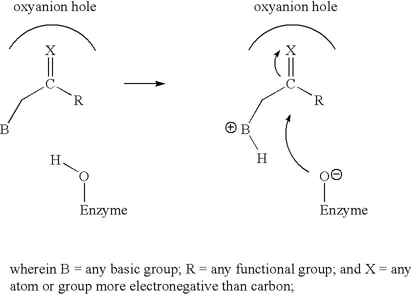

(2) the aspartic protease trigger involves the removal of a hydrogen from a water molecule to form a hydroxy anion, which can then attack the sp² as shown below:

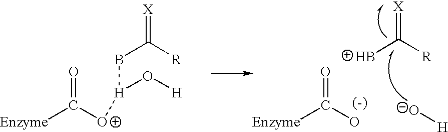

wherein X=an atom or group more electronegative than the sp² carbon; R any functional group; and B=any basic group

(3) the metalloprotease trigger involves the removal of a hydrogen from a water molecule to form a hydroxy anion, which can then attack the sp2 carbon as shown below:

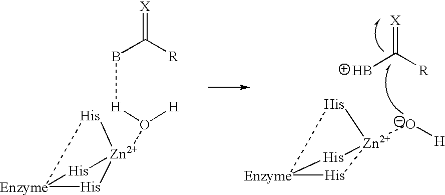

wherein X=an atom or group more electronegative than the sp² carbon; R=any functional group; and B=any basic group; and

(4) the cysteine protease trigger involves the addition of a proton to the X position (carbonyl in native substrate), which can augment or replace the oxyanion hole. This allows the cysteine to attack the carbon atom as shown below:

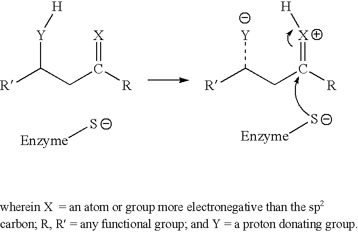

Example 2

### Determining the Trigger Mechanism for a Serine Protease

Quantum Core Technology™ has been used to identify reactive chemical moieties that triggers or inhibits a serine protease. Activators and inhibitors were desired to target an exemplary serine protease. The method employed for the process of identifying the chemical moieties is provided herein. These steps are the preferred methods for isolating the triggers of a serine protease and are presented for the mere purpose of teaching others skilled in the art to use the invention described herein and are not to be construed as limitations to the invention.

The first step of the QCT™ method is to develop a general classification of a chosen enzyme according to its reaction type. The amino acid sequence of the exemplary serine protease was procured from an enzyme database. A preferred database is Swiss Prot, although if the sequence for the exemplary serine protease was unknown, it could be determined through methods provided herein by identifying the DNA sequence and applying the genetic code dictionary.

The sequence of the exemplary serine protease sequence was aligned to other serine proteases. A source of known sequences, the Protein Data Bank at the Brookhaven National Laboratory possesses categories of known enzymes in the serine protease class. If it has been solved, the three-dimensional structure of the exemplary serine protease can also be found at the Protein Data Bank at the Brookhaven National Laboratory. If the structure of the exemplary serine protease has not been solved, it can be accomplished using methods known in the art of x-ray crystallography and/or nuclear magnetic resonance (NMR).

In order to derive the general category of chemical reaction that the exemplary serine protease undergoes, its three-dimensional structure was compared to those of other known serine proteases and found to be at least 20% homologous. A homology model of the exemplary serine protease was then constructed using the homology module of MSI Software Package (MSI Molecular Simulations, San Diego, Calif.) Although the homology module of MSI Software Package was the preferred method for constructing the homology model, there are other suitable programs including MDL, Tripos, and Oxford Molecular.

Once the model of the exemplary serine protease was created, it was analyzed using the MSI Software Package to extract the role of the exemplary serine protease's chemical functionalities. The simulation software provided a three-dimensional view of the serine protease of interest and the ability to enlarge the amino acid residues homologous to the known serine proteases. The catalytic residues of the active site of the exemplary serine protease were compared to the catalytic residues of the active sites of other known serine proteases to determine the mechanism of the protein of interest. In addition, the catalytic residues of the exemplary serine protease can also be analyzed by comparing the alignment of the amino acid sequences of various serine proteases and identifying the extent of conservation of specific catalytic residues at the active sites. Only amino acids that are conserved within all species of the serine proteases are the actual catalytic residues. Site-directed mutagenesis of the conserved catalytic residues can be performed to confirm that the conserved residues play significant roles in triggering the protease of interest. A preferred methodology for performing site-directed mutagenesis is discussed in Weiner, M. P., Gackstetter, T., Costa, G. L., Bauer, J. C., and K. A. Kretz, Molecular Biology: Current Innovations and Future Trends, A. M. Griffin and H. G. Griffin (eds), 1995 which is incorporated herein by reference.

Once the catalytic residues were ascertained, simple chemical reactions were derived comprising the catalytic residues of the active site and a chemical model was constructed. Construction of the chemical model was done by taking only the functional chemical groups and placing them in the proper spatial conformation, which is suggested by the homology model of the exemplary serine protease.

The serine protease mechanism involves charge relay between the three members of the catalytic triad, resulting in the activation of the serine nucleophile. A model demonstrating the trigger mechanism is as follows:

Resting State of the Serine Protease:

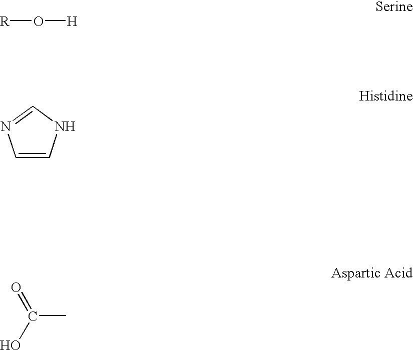

Substrate Introduced:

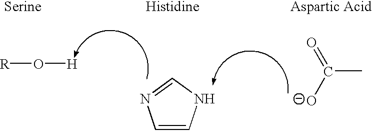

Substrate Released:

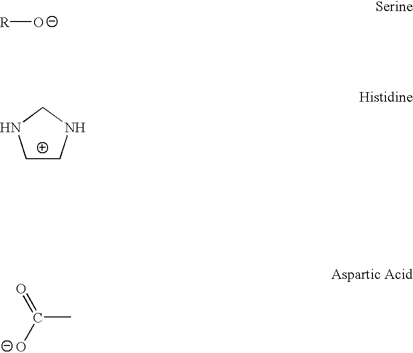

As one can see, the essence of the serine protease mechanism is the activation of the catalytic serine nucleophile by the extraction of a proton from its hydroxyl group, which forms an intensely charged hydroxyl anion. The formation of the charged hydroxyl anion results from a charge relay mechanism wherein the histidine extracts the proton from the hydroxyl of the serine and the aspartic acid residue extracts the hydrogen of the histidine, thereby making the histidine residue neutral. Furthermore, an additional factor that facilitates the nucleophilic attack of the hydroxyl anion on the carbonyl is the presence of an oxyanion binding site. The oxyanion site, which is depleted by the two amino groups, is a narrow hole in which the oxygen of the carbonyl of the substrate enters during the formation of the enzyme substrate complex. In the oxyanion binding site, multiple hydrogen bonds form with the oxygen of the substrate's carbonyl, thereby polarizing the C═O bond, which provides for a greater positive charge to the carbonyl of the substrate. The greater positive charge on the carbonyl further facilitates the nucleophilic attack of the hydroxyl anion of the serine residue to the carbonyl carbon of the substrate.

Because the mechanism of the exemplary serine protease was understood, “ab initio” calculations were made next for various combinations of reactants to determine the stabilization energies of each potential tetrahedral complex and therefore the trigger of the protease. The reactions involving a more negative stabilization energy are more likely to represent possible moieties for activators. The activating chemical functionalities provide the trigger mechanism of the serine protease because even an uncharged serine OH group can serve as a nucleophile and bypass the need for the charge relay process. As an example, “ab initio” calculations regarding the anionic transition complexes of exemplary serine proteases are illustrated in Table III below.

TABLE III
Ab initio 6-31 + G*/3-21G* calculated stabilization
energies Eₛₜ of Tetrahedral Complexes

| Substrate | Eₛₜ kcal/mol |
|---|---|
| H(NH₂)C═O | −22.7 |
| H(OH)C═O | −32.6 |
| H(CF₃)C═O | −59.9 |
| H(F)C═O | −50.5 |
| H(CN)C═O | −62.2 |
| HB(OH)₂ | −53.2 |
| F(OH)₂P═O | −61.7^{a} |

^{a}The Eₛₜ for the F(OH)₂P═O was estimated from the reaction equation: HO + F(OH)₂P═O → (HO)₂PO₂— + HF
An example for a typical reaction is the following: OH⁻ + H(NH₂)C═O → OH(H)(NH₂)C—O—

Using these results from Table III, all factors important to the stabilization of the transition state of the reaction or the rate determining step of the reaction were analyzed. The most important determination from this analysis occurred in the bond between the oxygen of the serine protease and the carbonyl carbon of the formamide. As this bond increased in strength, the catalysis of the serine protease was enhanced. Therefore, the trigger for serine protease is the activation of the hydroxyl group on serine to form a hydroxyl anion. Based upon the application of principles of organic chemistry and the above “ab initio” calculations, triggering chemical moieties were determined for the exemplary serine protease.

### EXAMPLE 3

### How Mechanism Based Drug Design Can Identify Reactive Molecules to Modulate Cysteine Proteases

The Quantum Core Technology™ described in the general methodology has also been employed to identify chemical functionalities that trigger or inhibit cysteine proteases. The following preferred methodology is for the mere purpose of teaching others skilled in the art to use the invention described herein. The following process has exemplary steps for identifying triggering functionalities for an exemplary cysteine protease.

The first step of the QCT™ method is to develop a general classification of a chosen enzyme according to its reaction type. The amino acid sequence of the exemplary cysteine protease was determined through using the database Swiss Prot. If the sequence for the exemplary cysteine protease had not been known, it could have been determined using techniques known to those skilled in the art. The cysteine protease sequence determined from the Swiss Prot database was then aligned to other cysteine proteases whose sequences were available in databases. The exemplary cysteine protease was determined to be homologous to other known cysteine proteases.

In order to derive the general category of chemical reaction that the exemplary cysteine protease undergoes, its three-dimensional structure was compared to those of other known cysteine proteases. A model was constructed for the exemplary cysteine protease with the structure of a cysteine protease that was at least 20% identical using the homology module of MSI Software Package (MSI Molecular Simulations, San Diego, Calif.). Although the homology module of MSI Software Package was the preferred method for constructing the homology model, there are other suitable programs including MDL, Tripos, Oxford Molecular, and Molecular Operating Environment. Once the model of the exemplary cysteine protease was created, it was analyzed using the MSI Software Package, which provided a three-dimensional structure of the protein and the ability to enlarge the amino acid residues homologous to the known cysteine proteases. The catalytic residues of the active site of the exemplary cysteine protease were compared to the catalytic residues of other known cysteine proteases to determine the mechanism of the protease. In addition, catalytic residues of the exemplary cysteine protease can also be analyzed by comparing the alignment of the amino acid sequences of various cysteine proteases and identifying the extent of conservation of specific catalytic residues at the active sites. Only amino acids that are conserved within all species of cysteine proteases are catalytic residues. Site-directed mutagenesis of the conserved catalytic residues can be performed to verify that the conserved residues play significant roles in the mechanism of the protease of interest. A preferred methodology for performing site-directed mutagenesis is discussed in Weiner, et al. (Weiner, M. P., Gackstetter, T., Costa, G. L., Bauer, J. C., and K. A. Kretz, Molecular Biology: Current Innovations and Future Trends, A. M. Griffin and H. G. Griffin (eds), 1995).

Once the catalytic residues were ascertained, simple chemical reactions were derived comprising the catalytic residues of the active site and a chemical modul was constructed. Construction of the chemical model was done by taking only the functional chemical groups and placing them in the proper spatial conformation, which was suggested by the homology model of the exemplary cysteine protease. “Ab initio” calculations for various combinations of reactants were then performed to describe the mechanism of the cysteine protease of interest.

The reaction mechanism of cysteine proteases is distinct over the reaction mechanism of serine proteases. In serine proteases, the activation of the nucleophile occurs by a consecutive charge relay mechanism, wherein the histidine pulls a proton from the hydroxyserine and subsequently the aspartic acid pulls a proton from the histidine. In cysteine proteases, the cysteine-histidine pair exist as an ion-pair in the resting state of the enzyme. Consequently, the cysteine is already charged. “Ab initio” calculations demonstrate how the sulfur group of the cysteine protease is very different from the hydroxy group of the serine protease in their nucleophilic attack on a carbonyl group (Arad, et al. “A simulation of the sulfur attach in the catalytic pathway of papain using molecular mechanics and semi-empirical quantum mechanics,” J Am Chem Soc, 112:491-502 (1990); Arad, et al., “Structural and mechanistic aspects of 3C protease from the picornavirus family,” J Chem Infor Comp Sci 33:345-349 (1993); Arad, et al., Organic Reactivity, Physical and Biological Aspects, 1995; Arad, et al. J Mol Modeling, (1996)). Reactions (i) and (iii), provided below, illustrate this difference where the lack of protonation on the carbonyl oxygen yields no tetrahedral structure.

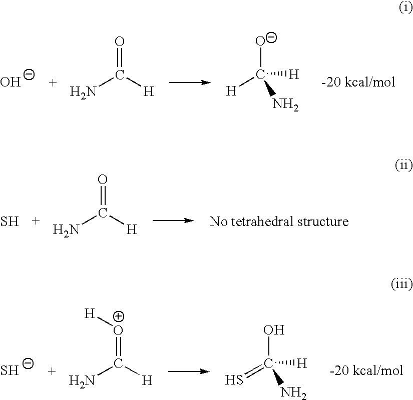

As in reaction (i), when the reaction of a hydroxy anion on formamide forms a charged tetrahedral structure, the product tetrahedral complex (TC) is 20 kcal/mol lower in energy (in the gas phase) than the sum of the reactants (the calculations are performed in the HF/6−31+G*//HF/6−31+G* level). The reaction with the suilfr (SH⁻) nucleophile on formamide has a completely different reaction potential surface. In this reaction (ii), the tetrahedral structure is not a minimum on the reaction potential surface. In order for the reaction to occur in a similar manner to the way it occurs in serine proteases, there has to be a protonation of the carbonyl. In reaction (iii), calculations performed on the same level (HF/6−31+G*//HF/6−31+G*) on the protonated set results in a similar reaction path to the one observed in serine proteases. In this ab initio study, it was shown that an early protonation of the substrate has to occur in order for the cysteine nucleophile to attack the peptide bond. Elsewhere (Arad, et al. “A simulation of the sulfur attach in the catalytic pathway of papain using molecular mechanics and semi-empirical quantum mechanics,” J Am Chem Soc, 112:491-502 (1990)), it was shown that the group that actually provides the proton necessary for protonation of the substrate is the catalytic histidine. Thus, a proton is transferred from the catalytic histidine to the substrate prior to the nucleophilic attack by cysteine.

Based on the above results, it appears that the catalytic histidine provides a proton which protonates the carbonyl of the substrate during the activation of the cysteine protease. This process enables the sulfur anion of the cysteine catalytic residue to attack the newly formed protonated carbonyl of the substrate, thereby forming the tetrahedral intermediate. The nucleophilic attack of the sulfur anion of the cysteine catalytic residue to the protonated carbonyl of the substrate is the rate determining step of the enzymatic reaction because this nucleophilic addition does not occur until the carbonyl of the substrate is protonated. The mechanism for cysteine protease thereby occurs as follows:

Resting State of the Cysteine Protease:

Charge Stabilization

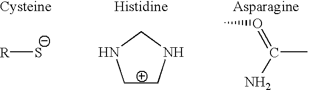

Substrate Introduced:

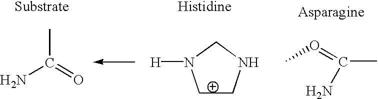

Interestingly, an oxyanion hole of the cysteine protease exists but is not sufficient to facilitate the nucleophilic attack of the sulfur anion to form the tetrahedral E−S structure. Therefore, full protonation of the substrate is needed. This protonation is done either by protonation of the carbonyl of the substrate or the nitrogen amide. In the instance where the nitrogen is protonated. the carbonyl is directed towards the oxyanion binding site. In the instance when the carbonyl is protonated, the carbonyl is directed towards the histidine.

In order for the reaction to proceed, the carbonyl oxygen must be protonated before the nucleophilic attack by the cysteine, which is indicated by the results shown in Table I. The neutral acetamide (column 5), which is most similar to the native substrate, does not form a stable complex with the cysteine sulfur (row 6). However, the protonated form of acetamide (column 6) does form a stable complex with the cysteine's sulfur (row 6). This table also indicates that the cysteine should be in the deprotonated form (row 6) rather than the neutral form (row 13) to form a stable complex. Thus, the protonation of the substrate and deprotonation of the active cysteine are prerequisites for the subsequent nucleophilic attack by the cysteine, which is in agreement with literature published about the reaction mechanism of the cysteine protease. Identifying substrate protonation as a trigger in the cysteine protease mechanism indicates that a functional motif capable of protonation would make a good activator. Making a compound with a hydroxy group close to the carbonyl would result in a self protonating compound by facilitating the transfer of a hydrogen from the carbonyl to the hydroxy group.

The QCT™ methodology further encompasses a means for tuning, or modulating, the reactivity of an inhibitor or activator. Having established that the relevant portions for designing an inhibitor of Table I are row 6 and the columns for protonated species, the relative energies of these compounds can be examined. Those compounds possessing stronger binding energies than possessed by acetamide, such as propan-2-one, will bind more strongly in the active site than the native substrate. Table I, thus, indicates a number of molecular motifs that could be present at the active site for enhanced binding ability. This is further supported and quantified by the computation of charge separations (Table IV-A) and orbital energies (Table IV-B).

With this information as guidance, a selection of structural motifs with some or all of these features can be computed, as shown in Table IV-C. Compounds of this size are expected to have some activity as inhibitors of many different cysteine proteases. The drug design process would then proceed to the addition of other functional groups that give specificity for a particular cysteine protease by binding to residues that are conserved across stereotypes, using the techniques of structure based drug design which are already well know to researchers in this field. The lead compounds could then be subjected to further testing for bioavailability and toxicity.

The procedures described in Examples 1 and 2 have been repeated for aspartic proteases and metalloprotease to demonstrate the universality of the method for determining chemical functionalities that trigger an enzymatic reaction. The trigger for the aspartic proteases is the activation of a water molecule to form a hydroxide anion. The trigger for the metalloproteases is the metal activation of the enzyme nucleophile. More importantly, the QCT™ methodology can be applied to any enzyme or protein—ligand interaction.

TABLE IV-A
Charge and bond length for cysteine and serine protease tetrahedral
complex calculations.
Table 1. Electronic and geometric characteristics of a stable
TC.

| | | Values of charge-transfer | | | Charges on reactive centers | | | | |
|---|---|---|---|---|---|---|---|---|---|
| | Tetrahedral | ab initio | ab initio | | Mullikan | | NAO | | |
| no. | complex (TC) | Mulliken | NAO | eq. 2 | X | A | X | A | X-A[a,b] |
| 1 | HO—CH₂—O⁻ | 0.6417 | 0.5563 | 0.3720 | −0.8337 | 0.2381 | −0.9257 | 0.3584 | 1.470 |
| 2 | HO—CF₂—O⁻ | 0.6053 | 0.6348 | 0.3883 | −0.8893 | 1.3604 | −0.8623 | 1.3537 | 1.396 |
| 3 | HO—CH₂—S⁻ | 0.7168 | 0.6261 | 0.4124 | −0.7728 | −0.0734 | −0.8708 | −0.2104 | 1.428 |
| 4 | HO—(CH)₂C—O⁻ | 0.6865 | 0.6107 | 0.3733 | −0.8002 | 0.4608 | −0.8818 | 0.6563 | 1.438 |
| 5 | HO—(NH₂)₂C—O⁻ | 0.6618 | 0.5823 | 0.2653 | −0.8283 | 0.6777 | −0.9073 | 0.9485 | 1.459 |
| 6 | HO—(NH₂)₂C—S⁻ | 0.7827 | 0.6401 | 0.3801 | −0.7229 | 0.1011 | −0.8654 | 0.4687 | 1.421 |
| 7 | HO—SiH₂—O⁻ | 0.4639 | 0.2914 | 0.4207 | −1.0296 | 1.1723 | −1.2062 | 1.9172 | 1.705 |
| 8 | HO—SiH₂—S⁻ | 0.5670 | 0.3140 | 0.4346 | −0.9354 | 0.8547 | −1.1943 | 1.4206 | 1.692 |
| 9 | HS—CF₂—O⁻ | 0.8109 | 0.8634 | 0.3623 | −0.2650 | 0.7802 | −0.2492 | 1.0459 | 1.915 |
| 10 | HS—CH₂—S⁻ | 0.8813 | 0.9634 | 0.3832 | −0.2286 | −0.4190 | −0.1702 | −0.7127 | 1.861 |
| 11 | HS—(NH₂)₂C—S⁻ | 0.8746 | 0.9669 | 0.3428 | −0.2160 | −0.0346 | −0.1640 | 0.1308 | 1.897 |
| 12 | HS—SiH₂—O⁻ | 0.5673 | 0.4821 | 0.3959 | −0.5042 | 0.8339 | −0.6445 | 1.5987 | 2.248 |
| 13 | HS—SiH₂—S⁻ | 0.6420 | 0.55731 | 0.4087 | −0.4476 | 0.6188 | −0.5814 | 1.0107 | 2.225 |

TABLE IV-B
Energies of the HOMO, LUMO, ionization
potential, and electron affinity obtained by “ab initio”
calculations.

| no. | Comp. | a_{HOMO} | a_{LUMO} | I[a] | A[a] |
|---|---|---|---|---|---|
| 1 | HO⁻ | −0.10333 | 0.41779 | 0.0589 | −0.3902 |
| 2 | HS⁻ | −0.09416 | 0.26090 | 0.0660 | −0.2444 |
| 3 | CH₂═O | −0.44295 | 0.07593 | 0.4046 | −0.0577 |
| 4 | CF₂═O | −0.55939 | 0.06644 | 0.5047 | −0.0536 |
| 5 | CH₂═S | −0.35227 | 0.03559 | 0.3373 | −0.0110 |
| 6 | (CH)₂C═O | −0.39286 | 0.05850 | 0.3593 | −0.0498 |
| 7 | (NH₂)₂C═O | −0.41446 | 0.06333 | 0.3762 | −0.1722 |
| 8 | (NH₂)₂C═S | −0.31813 | 0.04853 | 0.2911 | −0.0340 |
| 9 | SiH₂═O | −0.44595 | 0.02247 | 0.4075 | −0.0098 |
| 10 | SiH₂═S | −0.36239 | 0.00849 | 0.3396 | 0.0079 |

TABLE IV
C. Cysteine Protease Inhibitor Matrix
HF/6-31+G Geometry Optimized
in a.u. (hartrees)

| | | CH3S— TS | Binding | Protonated | CH3S— TC | Binding |
|---|---|---|---|---|---|---|
| Electrophile | Energy | (a.u.) | (kcal/mol) | Energy | Energy | (kcal/mol) |
| 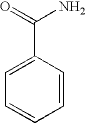 | −398.5 | NTC | | −398.8531 | −836.1716 | 120.2370953 |
| Benzamide | | | | | | |
| 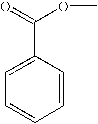 | −457.36 | NTC | | −457.6971 | −895.0231 | 124.9245913 |
| Benzoic acid | | | | | | |
| methyl ester | | | | | | |
| 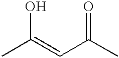 | −343.73 | NTC | | −344.0793 | −781.4099 | 127.8487855 |
| 4-Hydroxy- | | | | | | |
| pent-3-en-2-one | | | | | | |
| C4 attack | −343.73 | −780.8502 | −6.62023 | | | |
| 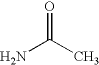 | −207.98 | NTC | | −208.3232 | −645.6607 | 132.1284003 |
| Acetamide | | | | | | |
| 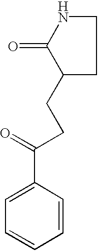 | −705.25 | NTC | | −705.5931 | −1142.933 | 133.8289511 |
| 3-(3-Oxo-3-phenyl-propyl)- | | | | | | |
| pyrrolidin-2-one | | | | | | |
| 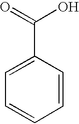 | −418.34 | NTC | | −418.6621 | −856.0038 | 134.7764904 |
| Benzoic acid | | | | | | |
| 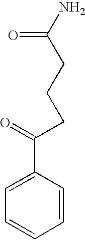 | −628.34 | NTC | | −628.6856 | −1066.033 | 138.2277927 |
| 5-Oxo-5-phenyl-pentanoic | | | | | | |
| acidamide | | | | | | |
| 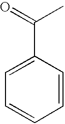 | −382.49 | NTC | | −382.8232 | −820.1745 | 140.8068567 |
| acetophenone | | | | | | |
| 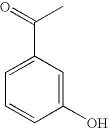 | −457.34 | NTC | | −457.6761 | −895.0318 | 143.5616234 |
| 3-hydroxy- | | | | | | |
| acetophenone | | | | | | |
| 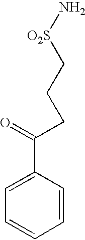 | −1062.8 | NTC | | −1063.092 | −1500.452 | 146.4544422 |
| 4-Oxo-4-phenyl-butane- | | | | | | |
| 1-sulfonic acid amide | | | | | | |
| 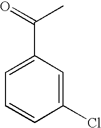 | −841.38 | NTC | | −841.7125 | −1279.073 | 146.774472 |
| 3-chloroacetophenone | | | | | | |
| 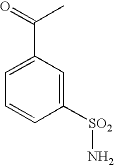 | −984.68 | NTC | | −985.0098 | −1422.371 | 147.0819517 |
| 3-Acetyl- | | | | | | |
| benzenesulfonamide | | | | | | |
| 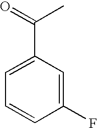 | −481.34 | NTC | | −481 6683 | −919.03 | 147.30158 |
| 3-fluoroacetophenone | | | | | | |
| 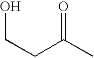 | −305.86 | NTC | | −306.1862 | −743.5496 | 148.3620711 |
| 4-Hydroxy-butan-2-one | | | | | | |
| zwatterion form | −305.86 | −742.9554 | −18.7814 | | | |
| 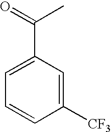 | −718.12 | NTC | | −718.4429 | −1155.807 | 148.7134764 |
| 3-trifluoromethyl- | | | | | | |
| acetophenone | | | | | | |
| 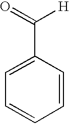 | −343.44 | NTC | | −343.77611 | −781.1431 | 150.6650309 |
| Benzaldehyde | | | | | | |
| 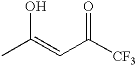 | −640.321 | −1077.45 | 4.8380981 | −640.63491 | −1078.002 | 150.8595589 |
| 1,1,1-Trifluoro-4-hydroxy- | | | | | | |
| pent-3-en-2-one | | | | | | |
| C4 attack | −640.32 | −1077.461 | 11.401851 | −640.6349; | −1077.993 | 144.722516 |
| 2-hydroxy-4-oxo | −640.31 | −1077.457 | 9.889551 | | | |
| C4 attack | −640.31 | −1077.442 | 0.081576 | | | |
| 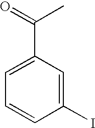 | −390.9 | NTC | | −391.2331 | −440.7548 | 154.6057906 |
| 3-iodoacetophenone | | | | | | |
| 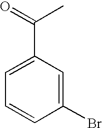 | −392.67 | NTC | | −392.9999 | −442.5245 | 156.4067429 |
| 3-bromoacetophenone | | | | | | |
| 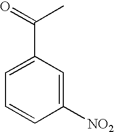 | −585.96 | NTC | | −586.2793 | −1023.657 | 157.2538807 |
| 3-nitro-acetophenone | | | | | | |
| 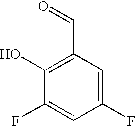 | −616.01 | −1053.151 | 10.15938 | −616.3176 | −1053.705 | 163.5415259 |
| 3,5-Difluoro-2-hydroxy- | | | | | | |
| benzaldehyde | | | | | | |
| C2 attack | −616.01 | −1053.121 | −8.60316 | −616.3176 | −1053.649 | 128.1186146 |
| zwitterion form | −615.98 | −1053.163 | 33.61568 | | | |
| 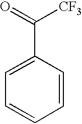 | −679.07 | −1116.205 | 6.595125 | −679.3724 | −1116.762 | 164.871846 |
| 2,2,2-Trifluoro- | | | | | | |
| 1-phenyl-ethanone | | | | | | |
| 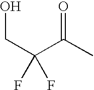 | −503.581 | −940.7133 | 5.867214 | −503.8801 | −941.2743 | 167.7207392 |
| 3,3-Difluoro-4-hydroxy- | | | | | | |
| butan-2-one | | | | | | |
| zwittenon form | −503.58 | −940.6984 | −3.53288 | | | |
| 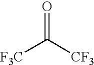 | −785.121 | −1222.2841 | 24.17794 | −785.3628 | −1222.831 | 214.1689924 |
| 1,1,1,3,3,3-Hexafluoro- | | | | | | |
| propan-2-one | | | | | | |

### EXAMPLE 4

### Determining Chemical Functionalities that Trigger the 3C Protease of the Cysteine Protease Family

The QC™ methodology has also been used to identify reactive chemical moieties that trigger or inhibit the 3C protease, a cysteine protease. The 3C protease is a versatile viral protease responsible for most cleavage events within the viral polyprotein and is considered to be indispensable in the reproduction of several virus types. Each step presented in the provided preferred methodology is for the mere purpose of teaching others skilled in the art to use the invention described herein.

The first step of the QCT™ method is to develop a general classification of a chosen enzyme according to its reaction type. The amino acid sequence of the 3C protease was determined using the preferred database, Swiss Prot. If the sequence for the 3C protease had not been known, it could have been determined by techniques known to those skilled in the art The 3C protease sequence determined from the Swiss Prot database was then aligned to other cysteine proteases to determine homology.

In order to derive the general category of chemical reaction that the 3C protease undergoes, its three-dimensional structure was compared to those of other known cysteine proteases, for example trypsin and kalekrein. Because there was at least 20% homology between the 3C protease and known proteins whose structure had been solved, it was concluded that a homology model could be constructed. The homology model was preferably constructed using the homology module of the MSI Software Package (MSI Molecular Simulations, San Diego, Calif.) Although the homology module of MSI Software Package was the preferred method for constructing the homology model, there are other suitable programs including MDL, Tripos, Oxford Molecular, and MOE. The active site of the model of 3C protease was analyzed using the MSI Software Package, which has the ability to enlarge select amino acid residues. To determine the mechanism for the 3C protease, the catalytic residues of the active site of the 3C protease were compared to the catalytic residues of other known 3C proteases. In addition, the catalytic residues of the 3C protease can also be analyzed by comparing the alignment of the amino acid sequences of various 3C proteases and identifying the extent of conservation of specific catalytic residues at the active sites. Only the amino acids that are conserved within all of the 3C proteases are the actual catalytic residues. Site-directed mutagenesis of the conserved catalytic residues can be performed to verify that the conserved residues play significant roles in the mechanism of the 3C protease. A preferred methodology for performing site-directed mutagenesis is discussed in Weiner, et al. (Weiner, M. P., Gackstetter, T., Costa, G. L., Bauer, J. C., and K. A. Kretz, Molecular Biology: Ciirretit Intovationis and Future Trends, A. M. Griffin and H. G. Griffin (eds), 1995).

Once the catalytic residues are ascertained, simple chemical reactions were derived comprising the catalytic residues of the active site. The chemical model was constructed by taking only the functional chemical groups and placing them in the proper spatial conformation, which was ascertained from the homology model. “Ab initio” calculations for various interactions were then performed to determine the mechanism of the 3C protease. The reactions resulting in a more negative stabilization energy are more likely to represent the reaction involving the trigger mechanism of the 3C protease. The trigger mechanism for 3C protease is similar to other cysteine proteases because the histidine must protonate the substrate for the reaction to proceed. Where the mechanism of the 3C protease differs from the mechanism of other cysteine proteases is that it does not contain an asparagine residue. The active site of the 3C protease consists of only two residues, cysteine and histidine. On the opposite side of the catalytic histidine, another conserved histidine at position 161 resides on the same side of the oxyanion binding site and functions either to protonate the carbonyl of the substrate or to direct the carbonyl to the histidine present in the catalytic site. The trigger mechanism for 3C protease thereby occurs as follows:

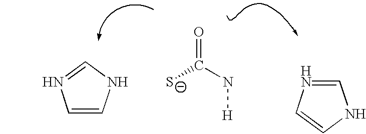

Because the mechanism of the 3C protease was understood, “ab initio” calculations were made next for various combinations of reactants to determine the stabilization energies of each potential tetrahedral complex and therefore the trigger of the protease. All of the examples thus far have considered simplistic “ab initio” calculations between one residue and a host of potential chemical moeities. Once a chemical group has been identified as being a potential activator or inhibitor, the effect of that chemical group can be considered with regard to other residues in the enzyme's active site.

For mechanism based drug design, it is very important to be able to compute, and thus understand how to modify, the reactivity of a small molecule that interacts with the enzyme active site. In order to compute reactivity in the active site of an enzyme, one must determine how much of the active site must be included to be predictive while keeping the calculation small enough to be performed routinely. Provided herein are a set of steps for doing this, along with illustrative data for the 3C protease active site, and a simple inhibitor, labeled cpi0016.

As was stated above, the trigger for substrate reactivity of the 3C protease is the transfer of a proton or formation of multiple hydrogen bonds to stabilize a carbonyl oxygen. Therefore, the deprotonated sulfur of a cysteine residue must attack the carbonyl carbon in the substrate for the reaction to proceed. Now that the trigger has been identified, portions of 3C protease substrates that could have a significant effect on the electronic properties at the attack site must be identified. For this illustrative example, the compound labeled cpi0016 is being used (common name 3′,5′-dichloro-2′-hydroxy-acetophenone) as a 3C protease substrate.

Next, a computable parameter that will vary according to the strength of the interaction between the enzyme and substrate portion must be identified. For this example, the bond length between the attacking cysteine sulfur atom of the 3C protease and the carbonyl carbon of cpi0016 will be used. Other suitable parameters might be bond angles, torsion angles, pyramidalization angles, bond energies, or atomic charges. The third step is to identify the portion of the active site that could be reasonably expected to affect the reactivity of the attack interaction. For this example, the crucial interaction is the stabilization of the carbonyl oxygen on cpi0016. This is done both by hydrogens on the 3C protease backbone and by the hydroxy group ion cpi0016 (an internal trigger). Becaise only the backbone is part of this interaction, the residue side chains were removed from the calculation, with the exception of the attacking cysteine. An “ab initio” calculation is then run on this portion of the system. For this example, geometry optimization calculations at the PM3 level of theory were used. Examine the C—S bond length of 2.000 Å. The complex being modeled is provided below:

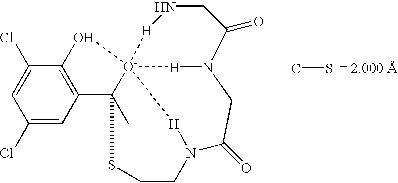

Next, the same calculation is performed using successively smaller portions of the 3C protease. For this example, the following results were obtained.

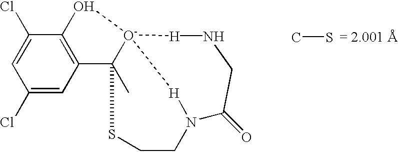

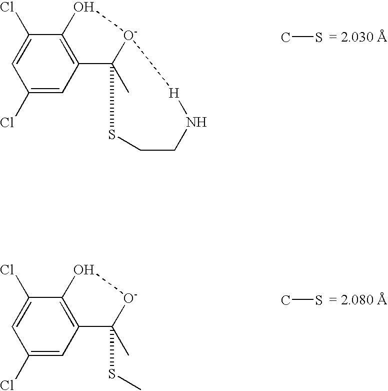

Once these calculations have been completed, they can be examined to determine how much of the enzyme must be included in the calculation at this stage. In this example, the second system, with a bond length of 2.001 Å0 is sufficient to obtain most of the electronic effect of the 3C protease. This same portion of enzyme could then be used in electronic structure calculations to test the electronic effect of various substrate groups. In general, a model system that recovers 90% of the electronic effect will reliably predict the electronic effects from the enzyme site.

Note that it will be necessary to test several computational levels of theory for performing the geometry optimizations and energy calculations, then choose the level of theory that gives an acceptable level of accuracy. The least time consuming calculations that give acceptable accuracy should be used. Some recommended levels of theory to test are PM3, HF/6−31+G*//PM3, B3LYP/6−31+G*//PM3, ONIOM(B3LYP/6−31+G*:UFF)

### EXAMPLE 5

### Identifying Compounds Using OCTTM and Employing Such Compounds to Screen for New Target Sites

Because the trigger for the 3C protease is a proton transfer to the substrate from the enzyme and the oxyanion binding site in 3C is not as tight as it is in serine or other cysteine proteases, a molecule with an internal hydrogen bond transfer capability is required per example of such a molecule is provided in the general scheme below:

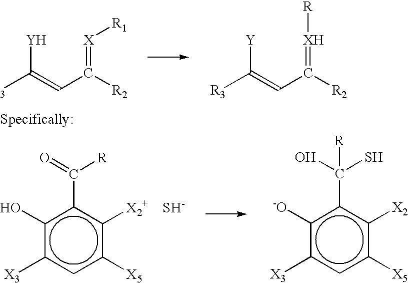

The “ab initio” calculations performed using the HF/6−31+G*//HF/6−31+G* basis presents the affinity for the internal proton transfer during a nucleophilic attack by a sulfur as a function of the addition of electron-withdrawing substituent X₃ and X₅ and as a function of R. The results are provided in Table V. In Table VI, also below, the original molecule and the addition product energies are calculated and presented. The energy difference between the reactants (original molecule+nucleophile) and the product is calculated. The larger the energy difference between the products and the reactants, the more stable the compound. The more stable the compound, the more likely it to serve as a candidate as a QCT lead molecule. For example, S3 has a stabilization energy of −16 kcal/mol. It is the best candidate because the value is most negative. The whole series of compounds were tested experimentally, and

TABLE V
Ab initio calculations for various combinations of reactants of a 3C
protease

| | Assi- | | | | | | | | | |
|---|---|---|---|---|---|---|---|---|---|---|
| | gned | | | | | | | | Energy of | Energy |
| Compound | num- | | | | | | | Energy | reactants | difference |
| name | ber | R | X5 | X4 | X2 | X3 | Basis set | (a.u.) | (a.u.) | (a.u.) |
| s1/ts1 | 6 | CH3 | H | H | H | H | 6 − 31 + G* | −457.35101 | −894.477 | 0.000503 |
| s2/ts2 | 9 | CH3 | H | H | OH | H | 6 − 31 + G* | −532.20479 | −969.34 | −0.00792 |
| s3/ts3 | 109 | CH3 | Cl | H | OH | Cl | 6 − 31 + G* | −1449.9938 | −1887.15 | −0.02554 |
| s4/ts4 | | CH3 | Cl | H | OH | H | 6 − 31 + G* | −991.10245 | −1428.25 | −0.01619 |
| s5a/ts5m | | CH3 | Am | H | H | Cl | 6 − 31 + G* | −1084.0271 | −1521.14 | 0.016818 |
| s5b/ts5m | | CH3 | Am | H | H | Cl | 6 − 31 + G* | −1084.038 | −1521.14 | 0.02769 |
| s5a/ts5 | | CH3 | Am | H | H | Cl | 6 − 31 + G* | −1084.0271 | −1521.2 | −0.04693 |
| s5b/ts5 | | CH3 | Am | H | H | Cl | 6 − 31 + G* | −1084.038 | −1521.2 | −0.03606 |
| s5a/ts5m1 | | CH3 | Am | H | H | Cl | 6 − 31 + G* | −1084.0271 | −1521.16 | −0.00572 |
| s5b/ts5m1 | | CH3 | Am | H | H | Cl | 6 − 31 + G* | −1084.038 | −1521.16 | −0.005154 |
| s4f1/ts4f1 | | CH3 | F | H | OH | H | 6 − 31 + G* | −631.05509 | −1068.19 | −0.01215 |
| scf3_1 | | CH3 | CF3 | H | OH | H | 6 − 31 + G* | −867.83234 | −1304.99 | −0.0262 |
| scf3-2 | | CH3 | H | H | OH | CF3 | 6 − 31 + G* | −867.83234 | −1304.98 | −0.02536 |
| SA | 44 | H | H | H | H | H | 3 − 21G* | −415.96604 | −851.034 | −0.02062 |
| SA* | 44 | H | H | H | H | H | 6 − 31 + G* | −418.30788 | −855.443 | −0.00821 |
| SA_033 | 33 | H | Cl | H | H | Cl | 3 − 21G* | −1329.6004 | −1764.7 | −0.05277 |
| SA_033 | 33 | H | Cl | H | H | Cl | 6 − 31 + G* | −32097.685 | −33283.1 | −748.313 |
| SA5MC | | H | Cl | H | H | Cl | 3 − 21G* | −872.78531 | | |
| SA_48 | 48 | — | H | H | H | CN | 3 − 21G* | −395.07915 | | 830.1266 |
| SA_48 | 48 | — | H | H | H | CN | 6 − 31 + G* | | | 437.1269 |
| SA_47 | 47 | H | I | H | H | I | 3 − 21G* | −14190.612 | | 14625.66 |
| SA_47 | 47 | H | Cl | H | H | Cl | 6 − 31 + G* | | | 437.1269 |
| SA_006 | 6 | CH3 | Cl | H | H | H | 3 − 21G* | | −889.87 | −454.823 |
| SA_006 | 6 | CH3 | Cl | H | H | H | 6 − 31 + G* | | | 437.1269 |
| SA3MC | | H | H | H | H | Cl | 3 − 21G* | −872.78291 | | 1307.83 |
| SA3MC | | H | H | H | H | Cl | 6 − 31 + G* | | | 437.1269 |
| SA5MC | | H | Cl | H | H | H | 3 − 21G* | −872.78531 | | 1307.833 |
| SA5MC | | H | Cl | | H | H | 6 − 31 + G* | −11412.291 | | 11849.42 |
| SA3CY | | H | H | H | H | CN | 3 − 21G* | −507.18284 | −942.282 | −0.05155 |
| SA3CY | | H | H | H | H | CN | 6 − 31 + G* | −14835.787 | −947.204 | −14323.63 |
| SA3CF3 | | H | H | H | H | CF3 | 3 − 21G* | −749.77977 | −1184.87 | −0.04469 |
| SA3CF3 | | H | H | H | H | CF3 | 6 − 31 + G* | | | 435.0475 |
| SA_SA3am | | H | H | H | H | CONH2 | 3 − 21G* | −582.8037 | −1017.89 | −0.03714 |
| SA_SA3am | | H | H | H | H | CONH2 | 6 − 31 + G* | | | |

TABLE VI
Calculations of Molecules to Screen for New Target Sites.

| Molecule | Energy E(a.u.) |
|---|---|
| 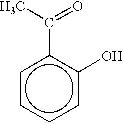 | −457.351005 |
| S1 | |
| 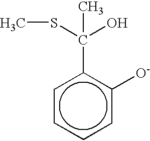 | −894.477378 |
| TS1 | ×E = 0.00051 |
| | =+ 0.32 kcal/mol |
| | −532.204792 |
| 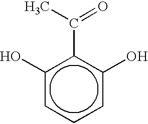 | −969.339584 |
| S2 | |
| 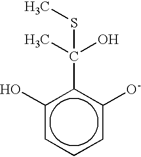 | −969.339584 |
| TS2 | ΔE = 0.00791 |
| | −4.96 kcal/mol |
| ^{⊖}S—CH₃ | −437.126876 |
| 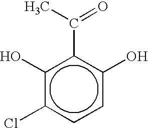 | −991.102451 |
| S4 | |
| 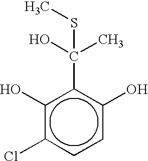 | −1428.245519 |
| TS4 | ΔE = 0.01617 |
| | −10.1 kcal/mol |
| 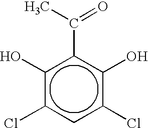 | −1449.993843 |
| S3 | |
| 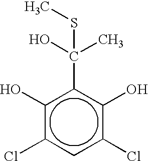 | −1887.14626 |
| TS3 | ΔE = 0.02562 = |
| | =−16.1 kcal/mol |
| 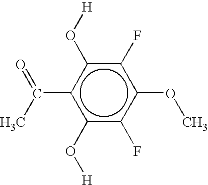 | −839.09848 a.u. (3-21G*) 3-21G* optimized |
| ^{⊖}S—CH₃ | −843.76074 a.u. |
| | 6-31+G*/+3-21G* |
| | −435.04747 a.u. |
| | 3-21G*, optim. |
| 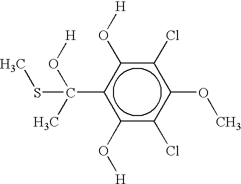 | −437.12688 a.u. 6-31+G*/3-21G* −1991.212728 a.u. 3-21G* |
| 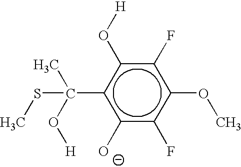 | −1932.82065 a.u. 6-31+G*/+3-21G* −1274.19992 a.u. 3-21G*, optimization |
| 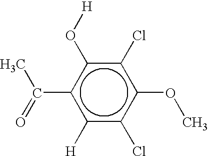 | −1280.91238 a.u. 6-31+G*/+3-21G* −1481.67055 a.u. 3-21G* |
| 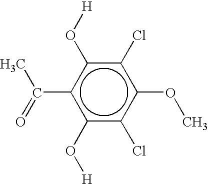 | −1556.10398 a.u. 3-21G*, optim |
| 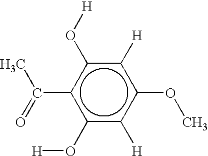 | −642.49668 a.u. 3-21G*, optim |
| 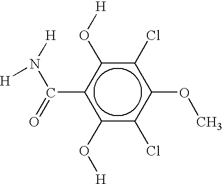 | −1572.030797 a.u. 3-21G*, opt |
| H₃C—S^{⊖} | −1916.73520 a.u. |
| 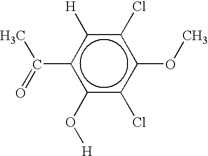 | 3-21G*, optim |
| 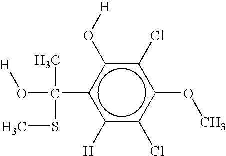 | −1916.77429 a.u. 3-21G*, optim |
| 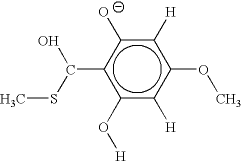 | 3-21G* −1077.5674247 |
| 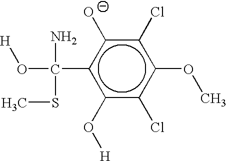 | 3-21G* −2007.111421 6-31+G* |
| CH₃—S^{⊖} | −1077.555814 |
| 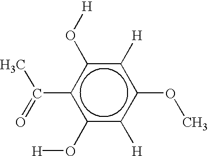 | |

there is a full correlation between the calculations and the experiment. Thus, as X₃ and X₅ more strongly withdraw electrons, the potency of the inhibitors increases.

### EXAMPLE 6

### Prophetic Examples of Pharmaceuticals Derived from Quantum Core Technology

Telomerase is a nucleoprotein expressed in immortalized cells, including most cancerous cells. Telomerase adds hexameric nucleotide repeats to telomeres, located at the ends of chromosomes to counteract the cellular aging process by which telomeres shorten at every cellular division. Because the majority of common human tumors possess telomerase activity, telomerase has become a universal cancer target. The two components of telomerase, the RNA component and the polymerase component, interact to form the active site. Sequences of both components are known although the structures are not. From the sequence identity, the telomerase polymerase is reported to be homologous to HIV reverse transcriptase, whose structure is known.

Telomerase inhibition can be accomplished through a variety of molecules reported in the scientific literature. The most important class of inhibitory molecules for the application in Quantum Core Technology™ are nucleotide analogs. “Ab initio” calculations can be made between the two components of the enzymes and the potential inhibitors and rank ordered according to the greatest negative Gibbs free energy, thereby determining the most likely inhibitory molecule to compete with the endogenous substrate. The structure of HIV reverse transcriptase can be used to model the hypothesized active site residues to interact with potential inhibitors. After determining the trigger mechanism of the target protein, a pharmaceutical agent comprising the isolate trigger can be synthesized.

In another prophetic example, molecules derived from QCT™ can be used to treat metastases which possess active cathepsins. Cathepsins are diverse intracellular proteases that can cleave substrates with a variety of active catalytic residues including cysteine, serine, or aspartic acid. Cathepsins are active at acidic pH and chiefly located in lysosomes. There is no known inhibitor of any cathepsin in mammals. Cathepsins can be endopeptidases or exopeptidases and in normal cells contribute to cellular metabolism. Secretion of cathepsins to the extracellular milieu, however, is associated with a variety of pathological states: arthritis, cancer, and Alzheimer's disease.

In healthy cells, cathepsin D is regulated by estrogen and appears to play a role in the renewal of newborn epithelial tissues. Cathepsin D is an aspartic protease whose upregulation is diagnostic and prognostic of cancer. One mechanism by which cathepsin D can promote metastases in the extracellular milieu is by liberating Fibroblast Growth Factor₂ (FGH₂), which consequently acts as a mitogen on cancer cells. From experiments performed in rats, it appears that cathepsin D can promote metastases at sites distal from the initial invasion by promoting growth and colonization. Cathepsin D can activate cathepsin B, which can further activate metastatic promoting protease cascades (see below). Another way in which cathepsin B can influence metastases is through competing with IGFII for receptor binding.

Cathepsin B is a cysteine protease whose upregulation and increased activity is associated with cancer diagnosis and prognosis. Its ability to degrade fibronectin, laminin, and collagen IV at physiological pH suggests that cathepsin B could act in cancer to promote metastases. Yet another demonstrated role for cathepsin B is activating a protease cascade that promotes metastasis. Procathepsin B can be activated by cathepsin D, which in turn can activate pro-urokinase-type plasminogen activator. Urokinase-type plasminogen activator converts plasminogen into plasmin, which is then capable of degrading the tumor stroma. Plasmin can also activate all three major matrix metalloproteases, each able to contribute to the degradation of the tumor stroma. Involvement in tumor angiogenesis might be another way in which cathepsin B promotes cancer.

Both cathepsins B and D appear to be upregulated in cancer and promote metastases through at least one common mode: their protease activities. The protease activities of both cathepsins B and D are excellent targets for inhibitor development using the quantum core technology. “Ab initio” calculations can be made between the cathepsins and the potential substrates and rank ordered according to the greatest negative Gibbs free energy, thereby determining the most likely inhibitory molecule. After determining the trigger mechanism of the target protein, a pharmaceutical agent comprising the isolate trigger can be synthesized.

In yet another prophetic example in which molecules derived from QCTT can be utilized is in reducing antibiotic resistance by inhibiting certain amidase activity in mycobacteria and other actinomycetes. Mycothiol is an antioxidizing molecule produced in actinomycetes, including mycobacteria, but not in humans. The presence of this protein in pathogenic organisms and absence in humans suggests that it could be a specific drug target, if its inhibition led to infection clearance with minimal side effects. In fact, when Mycobacterium smegmatis mutants are generated for decreased mycothiol biosynthesis, existing compounds like peroxide, superoxide, and the antibiotic rifampin are able to more effectively kill the organisms. These observations indicate that inhibiting mycothiol biosynthesis could render existing therapeutics more effective against mycobacteria infections.

A pathway for detoxifying antibiotics has been identified in mycobacteria in which mycothiol forms a thiol conjugate with an electrophilic moeity of an antibiotic. Mycothiol S-conjugate amidase (termed amidase-I) then cleaves the compound into a mercapturic acid which is secreted from the cell and glucosamine-alpha (1->1)-myo-inositol which is resynthesized into mycothiol. Amidase I has been identifed in the genomes of M. smegmatis and M. tuberculosis and a second protein with 36% identity to amidase I has been identified in M. tuberculosis, which represents a possible second protein involved in the detoxifying process. A streptomycete null mutant for armidase-I is more sensitive to killing by isoniazid, erythromycin, tetracycline, and vancomycin.

Amidase-1 inhibitors would severely impair mycothiol biosynthesis by reducing the necessary precursor. Without a functioning amidase-1, mycothiol will form a thiol conjugate with an antibiotic, but not undergo subsequent release. In this way the amount of mycothiol in each organism will decrease, making the organism vulnerable to antibiotics. Antibiotic resistance is on the rise throughout the world, and it is reasoned that a compound able to increase the lifetime utility of current therapies will enhance the repetoire of pharmacopoeia available to the clinician.

Amidase-1 is an excellent target for inhibitor development using QCT™. Amidase-1 can be cloned and expressed in great enough quantity to facilitate solving a crystal structure. After this step, “ab initio” calculations can be made between the amidase-1 and its potential substrates and rank ordered according to the greatest negative Gibbs free energy, thereby determining the most likely inhibitory molecule. After determining the trigger mechanism of the target protein, a pharmaceutical agent comprising the isolated trigger can be synthesized.

We claim:

1. A molecule comprising a minimal quantity of atoms to trigger a desired reaction wherein said molecule is designed by the following steps:
(a) determining the minimal quantity of atoms to trigger a desired reaction to occur, said minimal quantity of atoms collectively comprise the trigger mechanism for the desired reaction; and
(b) positioning said minimal quantity of atoms on a ligand wherein said molecule can trigger said desired reaction independently from the naturally occuring desired reaction.

2. A molecule comprising a minimal number of atoms for inhibiting a mechanism of action of a given reaction wherein said molecule is designed by the following steps:
(a) determining the trigger mechanism of the given reaction; and
(b) positioning said minimal number of atoms on a ligand wherein said ligand comprises an atom that functions as a poor leaving group when said molecule interacts with a reactant of the given reaction, thereby causing said molecule to inhibit the mechanism of action of the given reaction.

3. A molecule of claim 1 wherein said molecule comprises the minimum quantity of atoms to trigger the reaction of aspartic protease and has the following basic structure: 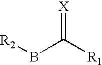
wherein B can be any basic group;
R₁ and R₂ can be any functional group; and
x can be any atom or group that is more electronegative than sp² carbon.

4 A molecule of claim 1 wherein said molecule comprises the minimum quantity of atoms to trigger the reaction of metallo protease and has the following basic structure: 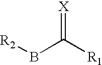
wherein B can be any basic group;
R₁ and R₂ can be any functional group; and
x can be any atom or group that is more electronegative than sp² carbon.

5. A molecule of claim 1 wherein said molecule comprises the minimum quantity of atoms to trigger the reaction of cysteine protease and has the following basic structure 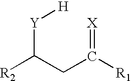
wherein x can be any atom or group that is more electronegative than sp² carbon;
R₁ and R₂ are any functional group;
and y is any proton donating group.

6. A molecule of claim 1 wherein said molecule comprises the minimum quantity of atoms to trigger the reaction of serine protease and has the following basic structure: 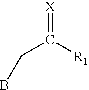
wherein B can be any basic group;
R₁ and R₂ can be any functional group; and
x can be any atom or group that is more electronegative than carbon.

7. A system for developing a small molecule wherein said system comprises:
(a) a method to compare the stabilization energies of a given reaction between a catalytic residue of an enzyme to residues of a substrate to determine the trigger mechanism for the given reaction,
(b) a minimal amount of atoms comprising said trigger mechanism of the given reaction; and
(c) a ligand wherein said minimal amount of atoms are positioned on said ligand to form said molecule.

8. A molecule that can inhibit a given reaction wherein said molecule is designed from the system in claim 7.

9. A molecule that can enhance the probability of a given reaction occuring wherein said molecule is designed from the system in claim 7.

10. A molecule wherein said molecule comprises a minimum quantity of atoms to inhibit the given reaction wherein said molecule is designed from the system in claim 7.

11. A molecule in claim 10 wherein said minimum quantity of atoms includes a poor leaving group to inhibit the given reaction.

12. A method for determining the trigger mechanism of a given reaction wherein said method uses a matrix involving the steps of:
(a) performing ab initio calculations on a class of enzymes to compare a reactive residue associated with a catalytic site common to the class of enzymes to a reactive residue with one or more substrates associated with the class of enzymes;
(b) using the information generated from step (a) and performing further ab initio calculations on a subclass of enzymes by comparing further reactive residues associated with the catalytic site common to the subclass of enzymes with one or more substates associated with the subclass of enzymes;
(c) using the information generated from step (b) and performing further ab initio calculations on a single enzyme from the subclass of enzymes by comparing all reaction residues associated with the catalytic site of the enzyme with one or more substrates associated with the enzyme; and
(d) using the information generated from step (c) to determine the trigger mechanism for the enzyme.

13. A method for determining a trigger mechanism for a given reaction comprising the steps of:
(a) performing ab initio calculations to determine the stability of each potential interaction between each active site residue of an enzyme and a chemical moiety of one or more substrates of said enzyme;
(b) analyzing the calculated stabilization energies, wherein the most negative energies are the most reactive species and the most positive energies are the least reactive species; and
(c) combining the chemical moieties that were calculated to be the most reactive, or the least reactive, with each corresponding catalytic residue on a compound scaffold.

14. A method for deteriming the trigger mechanism of a given reaction comprising the steps of
(a) aligning a protein sequence of an enzyme with other proteins and determining by homology the general class of the enzyme;
(b) performing biochemical assays to determine the reaction performed by the enzyme,
(c) performing site-directed mutagenesis of conserved amino acids and determining if the mutated residues act in the reaction mechanism;
(d) determining the mechanism of catalytic action for the enzyme; and
(e) performing ab initio calculations to determine the stability of each potential interaction between each active site residue of the enzyme and a chemical moiety of one or more substrates of said enzyme.

15. A method of designing a small molecule that interacts with the active site of a certain enzyme comprising the steps of.
(a) aligning the protein sequence of the enzyme with other proteins and determining by homology the general class of enzyme;
(b) performing biochemical assays to determine the reaction performed by the enzyme;
(c) performing site-directed mutagenesis of conserved amino acids and determining if the mutated residues act in the reaction mechanism;
(d) determining the mechanism of catalytic action for the enzyme;
(e) performing ab initio calculations to determine the stability of each potential interaction between each active site residue of the enzyme and a chemical moiety of one or more substrates of said enzyme;
(f) analyzing the calculated stabilization energies, wherein the most negative energies are the most reactive species and the most positive energies are the least reactive species; and
(g) combining the chemical moieties that were calculated to be the most reactive, or the least reactive, with each corresponding catalytic residue on a compound scaffold.

16. A molecule that can inhibit a given reaction wherein said molecule is designed from the methodology in claim 15.

17. A molecule that can enhance the probability of a given reaction occuring wherein said molecule is designed from the methodology in claim 15.

18. A molecule comprising a minimum quantity of atoms that function as a trigger mechanism for a given reaction wherein said molecule is designed from the methodology in claim 15.

19. A molecule comprising a minimal quantity of atoms to inhibit the activation of telomerase wherein said molecule comprises a poor leaving group.

20. A molecule comprising a minimal quantity of atoms to inhibit the activation of mycothiol s-conjugate amidase wherein said molecule comprises a poor leaving group.
